(19) United States
(12) Patent Application Publication
(10) Pub. No.: US 2006/0039931 A1
(43) Pub. Date: Feb. 23, 2006

(54) Stable cream preparations of phenyl-pyridone compounds for topical application

(75) Inventors: Max Werner Scheiwe, MAULBURG (DE); Yalcin Centikaya, Frenkendorf (CH)

Correspondence Address: HOFFMANN & BARON, LLP, 6900 JERICHO TURNPIKE, SYOSSET, NY, 11791, US

(73) Assignee: MEPHA AG, AESCH (CH)

(21) Appl. No.: 10/527,555

(22) PCT Filed: Sep. 11, 2003
(86) PCT No.: PCT/CH03/00615

§ 371 Date: Mar. 11, 2005

(30) Foreign Application Priority Data
Sep. 13, 2002 (CH) 155/02

(51) Int. Cl. A61K 31/4412; A61K 9/00

(52) U.S. Cl. 424/400; 514/345

(57) ABSTRACT

The invention relates to a pharmaceutical cream preparation for topical application in the form of an oil-in-water (o/w) emulsion, containing the following constituents in the lipophilic phase: (i) an optionally substituted 1-phenyl-2-(1H)-pyridone compound or a pharmaceutically acceptable salt thereof, as an active ingredient, (ii) at least one surface-active solubilising agent having an HLB value between 15 and 20, (iii) at least one emulsifier having an HLB value between 8 and 15, and (iv) optionally other carrier materials and additives known per se selected from the group containing triglycerides, penetration amplifiers, preserving agents and anti-oxidants. The invention also relates to the use of the preparation as a topical cream preparation for the treatment or prophylaxis of skin diseases.

The present invention relates to novel stable cream preparations for topical applications in which at least one optionally substituted phenylpyridone compound is present as the active ingredient. The preparations according to the invention are distinguished by good chemical and physical stability and are stable on storage. The cream preparations according to the invention are suitable for the medical treatment of skin diseases, especially those of a fibrotic nature.

The treatment of skin diseases of a fibrotic nature with emulsions containing optionally substituted phenylpyridones is described e.g. in U.S. Pat. No. 5,310,562 and EP 0 383 591. Thus, for example, pirfenidone [5-methyl-1-phenyl-2-(1H)-pyridone] has a broad spectrum of application in the treatment and prophylaxis of tissue and skin diseases such as fibrous lesions, pulmonary fibrosis, fibrosis of the prostate, scleroses, keloids, collagenoses, scar folds, postoperative adhesions, Alzheimer's disease, etc.

For the healing and prophylaxis of fibrous lesions, WO 97/41830 describes the use of substituted pyridones in different forms of administration such as capsules, tablets, powders, granules, syrups, injectable liquids, creams, ointments, inhalable liquids, eye drops, suppositories and pills.

WO 99/47140 discloses the use of optionally substituted phenylpyridones, e.g. pirfenidone, in pharmaceutically active, topical preparations such as ointments, creams or foams.

WO 00/16775 discloses gels for topical use in the treatment and prophylaxis of skin diseases of a fibrotic nature, said gels containing an optionally substituted phenylpyridone compound, especially pirfenidone. Creams and ointments are also mentioned in the introduction.

However, there is also a great need for stable, pharmaceutically active creams containing an optionally substituted pyridone compound for the treatment of fibrotic skin lesions. Creams are found to be particularly beneficial and cooling on damaged skin and in many cases are preferred to ointments or gels. Furthermore, hydrophilic creams moisturize the skin and exhibit caring properties. Moreover, creams are usually absorbed completely into the skin, whereas gels dry out on the skin surface and produce a film which in some cases is found to have adverse effects (feeling of tightness, cosmetic impairment due to scale-like structures, etc.).

However, experiments have shown that pyridone compounds, for example pirfenidone, are unsuitable for use in emulsions because these pyridone compounds behave as emulsion breakers, i.e. they destabilize the emulsion in which they are present. When an emulsion “breaks”, the oily and aqueous phases separate, leading to unwanted coalescence, i.e. a kind of confluence or coagulation of the constituents. This often results in a change in pH and may even cause the active ingredient to crystallize out. This disadvantageous property of pirfenidone is not discussed in said patents and patent applications and no teaching is offered to remedy the problem. Creams are merely cited as a possible topical form of application.

Creams are multiphase semisolid pharmaceutical forms. They are so-called “non-flowing” emulsions consisting of a lipophilic phase and a hydrophilic aqueous phase. They can contain the active ingredient dissolved or dispersed in the aqueous phase or in the oily phase.

To make therapeutically useful cream preparations, it is essential to overcome the tendency of pirfenidone to break the emulsion if the chemical and physical stability and the storage stability of its emulsion are to be assured. Stability data are a significant part of the authorization of pharmaceutical products by health authorities.

Typical stability parameters are the homogeneity of the formulation, the absence of coalescence of the emulsion droplets (no “coagulation”), a practically constant viscosity, semisolid structures, the complete dissolution of the active ingredient, and no subsequent crystallization of the active ingredient out of the emulsion.

Another requirement of pharmaceutically permissible formulations is that only auxiliary substances that are pharmaceutically acceptable and preferably described in the pharmacopoeias are used. Auxiliary substances that are not described in the pharmacopoeias must have their toxicological safety verified by toxicological studies, which are usually expensive. It is also necessary to verify that the patients' safety, i.e. the tolerability and efficacy of the drug in the therapeutic application, is assured.

Studies have shown that standard formulations of auxiliary substances, for example those described in the USP (United States Pharmacopoeia), are unsuitable for the preparation of pharmaceutically acceptable emulsions for topical applications in which pirfenidone is the active ingredient.

The object of the present invention is therefore to provide novel cream preparations containing pyridones as the active ingredient which retain their pharmaceutical efficacy and at the same time are chemically and physically stable, even under temperature stress, where applicable, and which have a good storage stability. It has now been found that, surprisingly, creams with the formulation indicated below have an outstanding stability.

The present invention provides a pharmaceutical cream preparation in the form of an oil-in-water (o/w) emulsion for topical application in the treatment and/or prevention of skin diseases, characterized in that said preparation contains the following constituents in the lipophilic phase:

(i) as the active ingredient, an optionally substituted 1-phenyl-2-(1H)-pyridone compound or a pharmaceutically acceptable salt thereof,

(ii) at least one surface-active solubilizer with an HLB value in the range 15-20,

(iii) at least one emulsifier with an HLB value in the range 8-15, and

(iv) optionally other excipients and additives known per se and selected from the group comprising triglycerides, penetration enhancers, preservatives and anti-oxidants.

The oil-in-water (o/w) emulsion preferably contains the oily phase in a proportion ranging from about 20 to 80% by weight and the aqueous phase in a proportion ranging from about 80 to 20% by weight.

Preferably, the oily phase is present in a proportion ranging from 24.1 to 84.1% by weight and the aqueous phase in a proportion ranging from 75.9 to 15.9% by weight; particularly preferably, the oily phase is present in a proportion ranging from 37.2 to 65% by weight and the aqueous phase in a proportion ranging from 35 to 62.8% by weight, based on the total weight of the preparation according to the invention.

The preparation or formulation according to the invention contains the active ingredient [component (i)] in the lipophilic phase preferably in an amount of 0.5-9% by weight and particularly preferably in an amount of 3-7% by weight, based on the total weight of the preparation.

The preparation or formulation according to the invention contains the surface-active solubilizer with an HLB value of 15-20 [component (ii)] preferably in a concentration of 5-65% by weight and particularly preferably in a concentration of 10-45% by weight, based on the total weight of the preparation.

The preparation or formulation according to the invention contains the emulsifier with an HLB value in the range 8-15 [component (iii)] preferably in a concentration of 3-30% by weight and particularly preferably in a concentration of 5-12.5% by weight, based on the total weight of the preparation.

The cream preparations contain as the active ingredient a substituted pyridone of general formula (I): 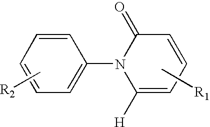
or a pharmaceutically acceptable salt thereof, in which R₁ and R₂ independently of one another can be (C₁-C₄)alkyl, carboxyl (—COOH) or —COOalkyl(C₁-C₄) and R₂ can also be hydrogen.

(C₁-C₄)alkyl R₁ and R₂ independently of one another are preferably methyl, ethyl, propyl, isopropyl, n-butyl, sec-butyl or t-butyl. If R₁ and/or R₂ are a radical —COOalkyl(C₁-C₄), the (C₁-C₄)alkyl radical therein has one of the meanings given above for R₁ and/or R₂.

Preferred substituted pyridones of general formula (I) are those in which R₁ is (C₁-C₄)alkyl and R₂ is hydrogen or (C₁-C₄)alkyl. The particularly preferred compound of formula (1) is that in which R₁ is methyl and R₂ is hydrogen (pirfenidone).

The salts known to those skilled in the art are to be regarded as pharmaceutically acceptable salts of the pyridone compounds of general formula (I), examples being the alkali metal and alkaline earth metal salts of the carboxyl-substituted compound of formula (I), preferably the sodium and magnesium salts, or the salts with oxalic acid, succinic acid, etc. of the compound of formula (I) that does not contain carboxyl.

The following preferred compounds may be mentioned as examples of representatives of the pyridones of formula (I) which can be present as the active ingredient in the cream preparations according to the invention:
5-methyl-1-p-tolyl-2-(1H)-pyridone
3-methyl-1-phenyl-2-(1H)-pyridone
3-ethyl-1-phenyl-2-(1H)-pyridone
4-isopropyl-1-phenyl-2-(1H)-pyridone
5-methyl-1-phenyl-2-(1H)-pyridone
3-methyl-1-carboxyphenyl-2-(1H)-pyridone
5-carboxy-1-phenyl-2-(1H)-pyridone
4-carboxymethyl-1-phenyl-2-(1H)-pyridone
5-t-butyl-1-(p-carboxyethylphenyl)-2-(1H)-pyridone.

5-Methyl-1-phenyl-2-(1H)-pyridone, known as pirfenidone, is the preferred active ingredient.

Substituted pyridones are known compounds and can be prepared by the conventional techniques known to those skilled in the art, for example the techniques described in U.S. Pat. No. 3,974,281.

The creams according to the invention are oil-in-water emulsions with an aqueous continuous phase. The surface-active solubilizers have an HLB value in the range 15-20 and preferably in the range 15-18. The emulsifiers used in the preparation according to the invention have an HLB value of 8 to 18 and preferably of 8-15. Those skilled in the art are familiar with the fact that the boundaries between surface-active solubilizers and emulsifiers overlap to some extent. The boundaries indicated here apply to the present invention.

Examples of suitable surface-active solubilizers with the indicated HLB values are diethylene glycol monoethyl ether, polyethylene/propylene glycol copolymers, cyclodextrins, glyceryl monostearates, e.g. Solutol HS 15 (macrogol 15-hydroxystearate from BASF, PEG-660 15-hydroxystearates), sorbitan esters, polyoxyethylenesorbitan acid esters, polyvinyl alcohol, sodium laurylsulfate (anionic), glyceryl monooleates, etc.

The following anionic and non-ionic emulsifiers are examples of possible emulsifiers with the indicated HLB values: anionic emulsifying waxes, cetyl alcohol, cetylstearyl alcohol, stearic acid, oleic acid, polyoxyethylene/polyoxypropylene block polymers, addition products of 2 to 60 mol of ethylene oxide and castor oil and/or hydrogenated castor oil, wool wax oil (lanolin), sorbitan esters, polyoxyethylenalkyl esters, polyoxyethylenesorbitan fatty acid esters or polyvinyl alcohol. Glycerol monooleate and stearic acid are preferred. Phospholipids, e.g. lecithin, are unsuitable as surface-active solubilizers or as emulsifiers within the framework of the present invention.

Possible triglycerides are medium-chain and high-molecular triglycerides. Medium-chain triglycerides are glycerol esters of fatty acids having only 6-12 carbon atoms, e.g. caprylic/capric acid triglyceride. High-molecular triglycerides are glycerol fatty acid esters with long-chain fatty acids. They are e.g. triglyceride mixtures prepared from different natural fats. It is preferable to use medium-chain triglycerides, especially caprylic/capric acid triglyceride.

Suitable penetration enhancers include e.g. isopropyl myristate, oleic acid, sodium laurylsulfate or 1,2-propanediol, the last of these being preferred.

Typical examples of preservatives are benzyl benzoates, benzoic acid, benzyl alcohol, benzalkonium chloride, N-cetyl-N,N,N-trimethylammonium bromide (Cetrimide, Merck), chlorhexidine, chlorobutanol, chlorocresol, iminourea, parabens such as methyl-, ethyl-, propyl- or butylparaben, sodium methylparaben, sodium propylparaben, potassium sorbate, sodium benzoate, sodium propionate, phenol, phenoxyethanol, phenylethyl alcohol, phenylmercuric acetate, phenyl-mercuric borate, phenylmercuric nitrates, sorbic acid or thiomersal (sodium ethylmercurithiosalicylate). Methylparaben, propylparaben, sodium methylparaben and sodium propylparaben are preferred.

Examples of antioxidants are sodium metabisulfite, alpha-tocopherol, ascorbic acid, maleic acid, sodium ascorbate, ascorbyl palmitate, butylated hydroxyanisole, butylated hydroxytoluene, fumaric acid or propyl gallate. The preferred antioxidant is sodium metabisulfite.

Examples of possible pH regulators are sodium hydroxide, hydrochloric acid, and buffer substances such as sodium dihydrogenphosphate or disodium hydrogen-phosphate.

The cream preparations can also contain other auxiliary substances and additives, e.g. superfatting agents, solvents, consistency regulators or hydrotropic agents, in order to improve the flow behaviour. The additives indicated above can be present individually or several substances from the same group can be present in a mixture.

Examples of suitable superfatting agents are decyl oleate, hydrogenated castor oil, light mineral oil, mineral oil, polyethylene glycol and sodium laurylsulfate.

Possible solvents are maize germ oil, cottonseed oil, groundnut oil, sesame oil, soya bean oil, ethyl oleate, glycerol, isopropyl myristate, isopropyl palmitate, polyethylene glycol or polypropylene glycol.

Examples of suitable consistency regulators are cetyl alcohol, cetyl ester wax, hydrogenated castor oil, microcrystalline waxes, non-ionic emulsifying waxes, beeswax, paraffin or stearyl alcohol.

Suitable hydrotropic agents are alcohols such as ethanol or isopropyl alcohol, or polyols such as glycerol.

Typical formulations of the cream preparations according to the invention contain
(a) 3-7% by weight of active ingredient
(b) 3-30% by weight of emulsifier
(c) 5-65% by weight of surface-active solubilizer
(d) 5-30% by weight of triglyceride
(e) 2-20% by weight of penetration enhancer
(f) 2-20% by weight of superfatting agent
(g) 3-30% by weight of consistency regulator
(h) 0.01-3% by weight of preservative
(i) 0.1-5% by weight of antioxidant
(j) 1-50% by weight of solvent
(k) purified water ad 100% by weight (i.e. 20-80% by weight and especially 15.9-75.9% by weight of water).

Preferred cream preparations of the invention contain
3-7% by weight of active ingredient
5-12.5% by weight of cetylstearyl alcohol
10-45% by weight of macrogol 15-hydroxystearate
7-20% by weight of medium-chain triglyceride
3-10% by weight of propanediol
3-10% by weight of decyl oleate
5-12.5% by weight of stearic acid
0.02-3% by weight of sodium methylparaben and sodium propylparaben
0.2-3% by weight of sodium metabisulfite
1-50% by weight of solvent
purified water ad 100% by weight.

The creams are prepared by melting the lipophilic constituents together and heating the melt to 60-80° C. in one apparatus, and simultaneously heating the aqueous phase to the same temperature in a separate apparatus. The aqueous phase is then incorporated into the oily phase and the mixture is emulsified until homogeneous and stirred until it forms a semisolid cream. The pH is preferably adjusted to 5-7.5.

The topical cream preparations according to the invention are suitable for the treatment or prophylaxis of skin diseases such as those described in WO 00/16775. They are particularly suitable for the treatment and prophylaxis of skin diseases of a fibrotic nature, e.g. fibrous lesions, multiple warts, contact dermatitis and keloids, for promoting the healing of burns and for postoperative wound care, etc.

The preparations according to the invention produce pharmaceutically active and cosmetically pleasing creams. They have good chemical and physical stability, both after preparation and after storage for 3-6 months or longer, so neither phase separation nor crystallization of the active ingredient occurs.

The Examples which follow illustrate the invention without however implying a limitation.

### COMPARATIVE EXAMPLES

### COMPARATIVE EXAMPLE 1

A hydrophilic ointment according to USP 23 (United States Pharmacopoeia) was prepared:

| Auxiliary substance | Amount |
|---|---|
| Polypropylene glycol | 12.0 g |
| Stearyl alcohol | 25.0 g |
| White petrolatum | 25.0 g |
| Methylparaben | 0.025 g |
| Propylparaben | 0.015 g |
| Sodium laurylsulfate | 10.0 g |
| Purified water | 27.9 g |

Pirfenidone was incorporated into this ointment base in amounts of 3.5, 5.0 and 10% by weight using the following procedure, which is according to the conventional technique as described in USP 23:

The stearyl alcohol and white petrolatum were melted on a steam bath and heated to about 75° C. The remaining constituents, including the pirfenidone, were added after they had been dissolved in water and also heated to 75° C. The mixture was stirred until it solidified. The finished ointment was then transferred to small plastic tubes with a threaded neck, and sealed in with a screw cap.

The preliminary stability test showed that the ointment was physically stable before it was subjected to the complete stability test, including the determination of chemical and physicochemical parameters.

After storage for 6 months under standard conditions (25° C.±2° C., 59% rh±5%, where rh denotes relative humidity), a phase separation occurred in the ointment preparation and the emulsion became inhomogeneous due to coalescence (i.e. the droplets flowed together or “coagulated”). Moreover, the active ingredient had crystallized out during storage in all three concentration samples containing 3.5% by weight, 5% by weight and 10% by weight of pirfenidone, respectively. Furthermore, some of the ointments lost so much viscosity that they liquefied.

The ointment formulation with pirfenidone, prepared according to USP 23, thus exhibits insufficient stability, both in respect of the ointment formulation itself and in respect of the active ingredient, and is unsuitable for pharmaceutical application.

### COMPARATIVE EXPERIMENT 2

A cream was prepared according to the following formulation:

| Constituent | Amount |
|---|---|
| Pirfenidone | 5.0 g |
| Propylene glycol | 5.0 g |
| Decyl oleate | 5.0 g |
| Medium-chain triglyceride | 10.0 g |
| Diisopropyl adipate | 5.0 g |
| Stearic acid | 5.0 g |
| Cetylstearyl alcohol | 5.0 g |
| Polyoxyethylene-40 stearate | 2.5 g |
| Sorbitan monostearate | 2.5 g |
| Sodium methylparaben | 0.2 g |
| Sodium propylparaben | 0.2 g |
| Purified water | 54.6 g |

The following constituents were melted on a steam bath and heated to 80° C., with slow stirring: decyl oleate, medium-chain triglyceride, diisopropyl adipate, stearic acid, cetylstearyl alcohol, polyoxyethylene-40 stearate and sorbitan monostearate. The remaining constituents, including the pirfenidone, were dissolved in water and also heated to 80° C. The hot aqueous solution was added to the melt, with vigorous stirring, and cooled to 30° C., with stirring. The finished cream was transferred to plastic tubes with a threaded neck, and sealed in with a screw cap. Part of it was transferred to tubes. Stability tests were carried out as described in Comparative Example 1.

Before the start of the tests, the preparation was homogeneous, i.e. stable. After storage for 6 months under standard conditions (25° C.±2+ C., 60% rh±5%), the cream had the following properties:

The pirfenidone content of the sample remained unchanged, i.e. the sample remained chemically stable.

The sample contained crystallized pirfenidone. The sharp-edged particles of crystallized active ingredient caused unacceptable scratch marks on application to the skin.

Due to this property, the cream preparation proved unsuitable for pharmaceutical applications and hence incapable of obtaining authorization from the health authorities.

### EXAMPLE 1

A cream according to the invention was prepared according to the following formulation:

| Constituent | Amount |
|---|---|
| Pirfenidone | 500 g |
| Solutol HS 15 | 2500 g |
| Polypropylene glycol | 500 g |
| Decyl oleate | 500 g |
| Medium-chain triglyceride | 1000 g |
| Stearic acid | 750 g |
| Cetylstearyl alcohol | 750 g |
| Sodium methylparaben | 20 g |
| Sodium propylparaben | 10 g |
| Purified water | 3470 g |

The cream preparation was made up as follows: The Solutol HS 15 (macrogol 15-hydroxystearate from BASF, a non-ionic solubilizer) was melted in a water bath at 75° C. The pirfenidone was added to the molten Solutol HS 15, with stirring until the solution was clear. The polypropylene glycol, decyl oleate, medium-chain triglyceride, stearic acid and cetylstearyl alcohol constituents were added to this solution and stirring was continued at 75° C.

The sodium methylparaben and sodium propylparaben were dissolved in 3470 g of purified water at 70° C. The melt containing the fats, and the aqueous solution, were mixed together at their respective temperatures, briefly evacuated and stirred for 15 minutes at 70° C. The mixture was then cooled to 45° C., with stirring, homogenized at this temperature for 5 minutes and cooled further to room temperature (≦25° C.). The cream obtained was transferred to tubes and had the following properties:

| | |
|---|---|
| Appearance: | white to colourless, homogeneous; |
| | no crystals |
| pH (potentiometric): | 6.2 |
| Viscosity (rotational viscometer, | 38,900 mPa · s |
| shear rate 11.8/s at 20° C.): | |
| Pirfenidone content (HPLC): | 101.2% of theoretical value |
| Content of impurities and | <0.1% |
| decomposition products (HPLC, | |
| 100% method): | |

Results of the Stability Tests:

The cream was initially homogeneous. Half of the samples were stored under standard conditions (25° C.±2° C., 60% rh±5%) and the other half at 31° C.+2° C., 70% rh±5%. They were stored for 6 months.

The samples stored under both conditions were still free of crystals after 6 months. The pH, viscosity, active ingredient content and decomposition products only showed the usual deviations within the limits of experimental error of the analytical method.

Thus the preparation of Example 1 proved stable and can be used as a pharmaceutically acceptable formulation.

### EXAMPLE 2

The following cream formulation was prepared analogously to Example 1:

| Constituent | Amount |
|---|---|
| Pirfenidone | 5.0 g |
| Solutol HS 15 | 10.0 g |
| Polypropylene glycol | 8.0 g |
| Decyl oleate | 3.0 g |
| Medium-chain triglyceride | 2.0 g |
| Stearic acid | 10.0 g |
| Cetylstearyl alcohol | 10.0 g |
| Sodium methylparaben | 0.2 g |
| Sodium propylparaben | 0.2 g |
| Purified water | 51.6 g |

The cream obtained had the following properties:

| | |
|---|---|
| Appearance: | white, homogeneous; no crystals |
| pH (potentiometric): | 6.4 |
| Viscosity (rotational viscometer, | 47,020 mPa · s |
| shear rate 11.8/s at 20° C.): | |
| Pirfenidone content (HPLC): | 100.8% of theoretical value |
| Content of impurities and | <0.1% |
| decomposition products (HPLC, | |
| 100% method): | |

Results of the Stability Tests:

The cream was initially homogeneous. Half of the samples were then stored under standard conditions (25° C.±2° C., 60% rh±5%) and the other half at 31° C.±2° C., 70% rh±5%. They were stored for 6 months.

The samples stored under both conditions were still free of crystals after 6 months. The pH, viscosity, active ingredient content and decomposition products only showed the usual deviations within the limits of experimental error of the analytical method.

The cream preparation of Example 2 is stable and can be used as a pharmaceutically acceptable formulation.

### EXAMPLE 3

The following cream formulation was prepared analogously to Example 1:

| Constituent | Amount |
|---|---|
| Pirfenidone | 5.0 g |
| Solutol HS 15 | 22.0 g |
| Polypropylene glycol | 3.0 g |
| Decyl oleate | 12.0 g |
| Medium-chain triglyceride | 9.0 g |
| Stearic acid | 4.5 g |
| Cetylstearyl alcohol | 4.5 g |
| Sodium methylparaben | 0.2 g |
| Sodium propylparaben | 0.1 g |
| Sodium metabisulfite | 1.0 g |
| Purified water | 39.7 g |

In the preparation of the cream, sodium metabisulfite was dissolved together with sodium methylparaben and sodium propylparaben in water at 70° C. The procedure was otherwise as in Example 1.

The cream obtained had the following properties:

| | |
|---|---|
| Appearance: | white, homogeneous; no crystals |
| pH (potentiometric): | 6.3 |
| Viscosity (rotational | 41,256 mPa · s |
| viscometer, | |
| shear rate 11.8/s at 20° C.): | |
| Pirfenidone content (HPLC): | 99.7% of theoretical value |
| Content of impurities and | <0.1% |
| decomposition products | |
| (HPLC, 100% method): | |

Results of the Stability Tests:

The cream was initially homogeneous. Half of the samples were then stored under standard conditions (25° C.±2° C., 60% rh±5%) and the other half at 31° C.±2° C., 70% rh±5%. They were stored for 6 months.

The samples stored under both conditions were still free of crystals after 6 months. The pH, viscosity, active ingredient content and decomposition products only showed the usual deviations within the limits of experimental error of the analytical method.

The cream preparation of Example 3 is stable and suitable for pharmaceutical application.

1. Pharmaceutical cream preparation in the form of an oil-in-water (o/w) emulsion for topical application in the treatment and/or prevention of skin diseases, characterized in that said preparation comprises the following constituents in the lipophilic phase:
(i) as the active ingredient, an optionally substituted 1-phenyl-2-(1H)-pyridone compound or a pharmaceutically acceptable salt thereof,
(ii) at least one surface-active solubilizer with an HLB value in the range 15-20,
(iii) at least one emulsifier with an HLB value in the range 8-15, and
(iv) optionally other excipients and additives and selected from the group comprising triglycerides, penetration enhancers, preservatives and antioxidants.

2. Preparation according to claim 1, characterized in that it comprises the oily phase in a proportion ranging from 20 to 80% by weight and the aqueous phase in a proportion ranging from 80 to 20% by weight, based on the total weight of the preparation according to the invention.

3. Preparation according to claim 1, characterized in that it comprises the oily phase in a proportion ranging from 24.1 to 84.1% by weight and the aqueous phase in a proportion ranging from 75.9 to 15.9% by weight, based on the total weight of the preparation.

4. Preparation according to claim 1, characterized in that it comprises the active ingredient in an amount of 0.5-9% by weight and based on the total weight of the preparation.

5. Preparation according to claims 1-4, characterized in that it comprises the surface-active solubilizer in a concentration of 5-65% by weight based on the total weight of the preparation.

6. Preparation according to claim 1, characterized in that it comprises the emulsifier in a concentration of 3-30% by weight based on the total weight of the preparation.

7. Preparation according to claim 1, characterized in that it comprises as the active ingredient a substituted pyridone of general formula (I): 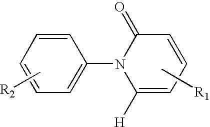
or a pharmaceutically acceptable salt thereof, in which R₁ is one of (C₁-C₄)alkyl, carboxyl(-COOH) or —COOalkyl(C₁-C₄) and R₂ is one of (C₁-C₄)alkyl, carboxyl(-COOH), —COOalkyl(C₁-C₄) and hydrogen.

8. Preparation according to claim 7, characterized in that when present (C₁-C₄)alkyl and alkyl((C₁-C₄) of R₁ and R₂ are independently selected from the group consisting of methyl, ethyl, propyl, isopropyl, n-butyl, sec-butyl, and t-butyl.

9. Preparation according to claim 7, characterized in that it contains as the active ingredient a compound of formula (I) in which R₁ is (C₁-C₄)alkyl and R₂ is hydrogen or (C₁-C₄)alkyl.

10. Preparation according to claim 1, characterized in that the active ingredient as is a pharmaceutically acceptable salt as an alkali metal or alkaline earth metal salt of the carboxyl-substituted compound of formula (I), or a salt of the compound of formula I which does not contain a carboxyl group with oxalic acid or succinic acid.

11. Preparation according to claim 1, characterized in that it comprises one of the following compounds as the active ingredient:
5-methyl-1-p-tolyl-2-(1H)-pyridone
3-methyl-1-phenyl-2-(1H)-pyridone
3-ethyl-1-phenyl-2-(1H)-pyridone
4-isopropyl-1-phenyl-2-(1H)-pyridone
5-methyl-1-phenyl-2-(1H)-pyridone
3-methyl-1-carboxyphenyl-2-(1H)-pyridone
5-carboxy-1-phenyl-2-(1H)-pyridone
4-carboxymethyl-1-phenyl-2-(1H)-pyridone
5-t-butyl-1-(p-carboxyethylphenyl)-2-(1H)-pyridone.

12. Preparation according to claim 1, characterized in that the surface-active solubilizer is selected from the group consisting of diethylene glycol monoethyl ether, polyethylene/propylene glycol copolymers, cyclodextrins, glyceryl monostearates, sorbitan esters, polyoxyethylenesorbitan acid esters, polyvinyl alcohol, sodium laurylsulfate (anionic) and glyceryl monooleates.

13. Preparation according to claim 1, characterized in that the emulsifier is selected from the group consisting of anionic and non-ionic emulsifiers, anionic emulsifying waxes, cetyl alcohol, cetylstearyl alcohol, stearic acid, oleic acid, polyoxyethylene/polyoxypropylene block polymers, addition products of 2 to 60 mol of ethylene oxide and castor oil and/or hydrogenated castor oil, wool wax oil (lanolin), sorbitan esters, polyoxyethylenalkyl esters, polyoxyethylenesorbitan fatty acid esters and polyvinyl alcohol.

14. Preparation according to claim 1, characterized in that the triglyceride is selected from the group consisting of medium-chain and high-molecular triglycerides.

15. Preparation according to claim 1, characterized in that the penetration enhancer is selected from the group consisting of isopropyl myristate, oleic acid, sodium laurylsulfate and 1,2-propanediol.

16. Preparation according to claim 1, characterized in that it also comprises at least one superfatting agents, solvents, consistency regulators and/or hydrotropic agents.

17. Preparation according to claim 1, characterized in that it comprises the following components:
(a) 3-7% by weight of active ingredient
(b) 3-30% by weight of emulsifier
(c) 5-65% by weight of surface-active solubilizer
(d) 5-30% by weight of triglyceride
(e) 2-20% by weight of penetration enhancer
(f) 2-20% by weight of superfatting agent
(g) 3-30% by weight of consistency regulator
(h) 0.01-3% by weight of preservative
(i) 0.1-5% by weight of antioxidant
(k) 1-50% by weight of solvent
(l) purified water balance to 100% by weight.

18. Preparation according to claim 1, characterized in that it comprises the following components:
3-7% by weight of active ingredient
5-12.5% by weight of cetylstearyl alcohol
10-45% by weight of macrogol 15-hydroxystearate
7-20% by weight of medium-chain triglyceride
3-10% by weight of propanediol
3-10% by weight of decyl oleate
5-12.5% by weight of stearic acid
0.02-3% by weight of sodium methylparaben and sodium propylparaben
0.2-3% by weight of sodium metabisulfite
1-50% by weight of solvent
purified water ad balance to 100% by weight.

19. Process for the production of a preparation according to claim 1, characterized in that the lipophilic constituents are melted together and the melt is heated to 60-80° C. in one apparatus, and the aqueous phase is heated to the same temperature in a separate apparatus, the aqueous phase is then incorporated into the oily phase and the mixture is emulsified until homogeneous and stirred until it forms a semisolid cream, the pH optionally being adjusted to 5-7.5.

20. (cancelled)

21. A method comprising treatment or prophylaxis of a skin disease selected from diseases of a fibrotic nature, fibrous lesions, multiple warts, contact dermatitis, and keloids or promoting the healing of burns or post-operative wound care comprising applying the preparation of claim 1 to skin.
